(19) United States
(12) Patent Application Publication
(10) Pub. No.: US 2017/0219455 A1
(43) Pub. Date: Aug. 3, 2017

(54) In-Line Composition and Volumetric Analysis of Vent Gases and Flooding of the Annular Space of Flexible Pipe

(71) Applicant: Schlumberger Technology Corporation, Sugar Land, TX (US)

(72) Inventors: Lars Nicolas Mangal, Croissy Sur Seine (FR); Stephane Vannuffelen, Meudon (FR); Rogerio Tadeu Ramos, Eastleigh (GB); Ricardo R. Vasques, Bailly (FR); Michel V. Berard, Paris (FR); Dominique Dion, Plaisir (FR)

(73) Assignee: Schlumberger Technology Corporation, Sugar Land, TX (US)

(21) Appl. No.: 15/489,694

(22) Filed: Apr. 17, 2017

Related U.S. Application Data
(63) Continuation of application No. 12/864,497, filed on Feb. 21, 2011.
(60) Provisional application No. 61/099,585, filed on Sep. 24, 2008.
(60) Provisional application No. 61/023,738, filed on Jan. 25, 2008.

(51) Int. Cl. G01M 3/28; G01F 1/84; G01N 1/10; G01N 30/02; E21B 17/01; G01N 33/00; E21B 47/10; E21B 49/08; E21B 47/06; E21B 34/00; G01F 1/68; G01N 35/00

(52) U.S. Cl. CPC G01M 3/2815; G01F 1/68; G01F 1/84; G01N 1/10; G01N 30/02; G01N 35/0092; G01N 33/004; G01N 33/0044; E21B 47/1025; E21B 49/08; E21B 47/06; E21B 34/00; E21B 47/065; E21B 17/01; E21B 2049/085

(57) ABSTRACT

A method and system for monitoring a flexible pipe, including an inline sensor system coupled to the annulus of the flexible pipe to detect corrosion of the flexible pipe. Also disclosed are method and system for monitoring an amount of water being accumulated in an annulus of a flexible pipe, including locating a pressure measurement system proximate to the annulus for measuring pressure of gas inside the annulus; controlling a flow of vent gas with a vent gas valve; positioning a flow measurement system upstream or downstream of the vent gas valve for measuring the flow of the vent gas when the vent gas valve is opened; and collecting with a microprocessor pressure and flow measurement data from the pressure and the flow measurement systems for determining the amount of water accumulated in the annulus based on the collected pressure and flow measurement data.

### BACKGROUND OF THE INVENTION

Field of the Invention

The present invention generally relates to the monitoring of pipe structures, and more particularly to a system and method for composition and volumetric analysis of vent gasses and detection of water flooding in an annular space of a flexible pipe structure.

Discussion of the Background

The monitoring of pipe structures is of great importance in many areas, in particular in the oil and gas industry, even more important in subsea environment where access to the structures is difficult. As an example, a pipeline running at the sea bed between an offshore production location to a transportation hub may need to be monitored to provide information regarding its integrity. Subsea production is growing in importance for many oil companies and is projected to increase significantly in the next 5-10 years. In addition, offshore fields are being exploited in deeper and deeper waters. However, producing from floating production platforms (FPSO) presents many challenges which increase as the water depth increases. Often, produced fluids are carried from the wellheads on the seabed to the FPSO through flexible risers or flow lines. Flexible risers bring many advantages allowing produced fluids to flow from the fixed seabed to that FPSO structure that will move with tidal and wave action. In addition, flexible risers can be manufactured in long continuous lengths, which allow for a simpler and more efficient installation. The use of flexible risers is well documented in many publications (see, e.g., Felix-Henry, A., “Prevention and Monitoring of Fatigue-corrosion of Flexible Risers' Steel Reinforcements,” OMAE2007-29186, Proceeding of the 26^{th} International Conference on Offshore Mechanics and Arctic Engineering, Jun. 10-15, 2007, San Diego, Calif., USA, incorporated by reference herein).

The monitoring of flexible pipes used in subsea applications, such as production risers, jumpers or flowlines, is necessary to avoid potentially catastrophic incidents like hydrocarbon spills, loss of well control, or escape of high amounts of gas that can affect the buoyancy of floating vessels, among others. The industry is currently using several techniques to identify damage in flexible pipes, and at least some such techniques are described in the document entitled, “Guidance note on monitoring methods and integrity assurance for unbonded flexible pipe,” prepared by UKOOA, Rev. 05, October 2002, and herein incorporated by reference. However, the methods disclosed in this document, as well as commonly used in the industry are time consuming and require production to be partially or totally stopped.

### SUMMARY OF THE INVENTION

The present invention includes the recognition that the methods and techniques commonly used in the industry for monitoring flexible pipe structures are time consuming and require the production of hydrocarbons to be partially or totally stopped. Because of the time inherent in these operations, they are performed infrequently and at great expense to the operators. This is the main driver to find innovative ways to permanently monitor the integrity of pipe's structures that are less intrusive resulting in an increase of the frequency of inspection to ultimately reduce the number of incidents.

According to the above mentioned document prepared by UKOOA, the highest number of failure incidents seen to date in the UK and Norwegian sector are annulus flooding, damage to the external sheath and degradation of the internal pressure sheath. The repercussions of the above mentioned failure incidents could result in corrosion and/or corrosion-fatigue type failure of the pipe structure. One method described in the aforementioned document is laboratory analysis done on samples of gases taken from the vent port of the annulus of flexible pipes. However, as noted above, this is a time consuming process.

Therefore, there is a need for a method and apparatus (e.g., which also can be referred to herein as a “system”) that addresses the above and other problems. The above and other needs and problems are addressed by a first exemplary embodiment of the present invention, which provides a method and apparatus for monitoring of pipe structures, particularly flexible pipes, including a systematic permanent monitoring and analysis of such gases via one or more in-line sensors (e.g., in-line spectrometers) coupled with a software interface that records the level of produced gas and the type of the gas and alerts the user if such levels are above the normal or acceptable limit. This novel method can be used to actively monitor gases resulting from the chemical reactions when the metal of the armor wires (also referred to herein as “armor wire layer” or “armor wire layers”) or pressure vault layer or any other metal portion of a flexible pipe, e.g., flexible riser, or other structure corrodes. Changes of the flowrate of produced gas can be inferred as degradation of the pressure sheath that allows gas and fluid from the production fluid to fill the annulus, and presence of water vapor indicates an annulus flooded with sea water. An exemplary aspect of the embodiment includes one or more inline sensors connected to a vent port of a flexible pipe and coupled with a data recording unit using a software interface to monitor and record levels and types of produced gases. Advantageously, the level and the type of produced gases can be analyzed with the software to identify if the integrity of the pressure sheath has been compromised, whether there is sea water entry in the annulus, whether the armor wire layer or pressure vault layer is corroding, whether the outer sheath has been damaged, other failure drivers, and the like.

A second exemplary embodiment of the present invention that can be optionally combined with the first exemplary embodiment, includes novel techniques for determining the presence of water or liquids in the annulus of a flexible pipe, e.g., flexible riser or flowline in a subsea system using information on the annulus gas vent rate and pressure. The presence of such water/liquid in the annulus can result from, for example, condensation in the annulus, slow intrusion of liquid from the inside or outside of the flexible pipe through the protective layers or break-down of an outer sheath of the flexible pipe, resulting in flooding of the annulus. Advantageously, information on variations of temperature in the riser annulus also can be provided. In an exemplary aspect, a device, including a pressure transducer together with a volumetric or mass flow meter, is placed in-line with the annulus vent port of a flexible riser. The device can be used to collect pressure, temperature and flow information during the gas annular venting event. In a further aspect, the device is located inside the end-fitting or before the annulus venting check valve (which may also be referred to herein as “vent-check valve” or “vent gas valve”), enabling the recording and interpretation data not only during the venting, but also during the build-up of the annular pressure. As pressures builds inside the annulus, it eventually reaches the pressure by which the vent check-valve in the end-fitting opens, resulting in gas being vented out of the annulus. A state-of the art vent check-valve includes a mechanical spring with a face seal remaining open until the pressure decreases to a pre-determined value at which time the valve closes and the annular pressure starts building again due to the constant but slow process of diffusion through the flexible pipe, e.g., flexible riser pressure sheath. During the initial venting period, the vented gas flow rate is typically significantly larger than the diffusion rate and dominates the pressure drop in the annulus space. The interpretation of the pressure drop and flow rate enables the determination of the total gas volume present in the annular space. If the diffusion rate is such that the vent check-valve does not generate sufficient flow rate during the initial stages of the venting sequence, a piloted valve can be used to increase the flow port opening and resulting flow rate. The later part of the draw-down and the build-up can be used to determine the diffusion rate of the particular riser.

The ability to accurately measure the total volume without the need to use a positive displacement meter advantageously is provided. Due to various vent system implementations, there is a wide range of flow rates that a meter must accurately cover, leading to the current practice of a positive displacement measurement system. However, this incurs additional maintenance and reduced service life due to the dynamic seals in the pistons. Accordingly, in a further aspect a flow meter can be employed with non-moving parts (e.g., an intrusive acoustic flow meter) with an additional valve to shut-off the flow when the flow rates cross a threshold below which the accuracy of the meter would be degraded. A further aspect replaces the vent check-valve by an actuated valve, enabling better control of the flowing rates and pressures to maximize the accuracy of the interpretation results. The ability to control the venting operation also enables a more efficient sample collection, whenever it is deemed necessary. Advantageously, variations in the temperature of the annulus can also be detected. The exemplary embodiments also can be used with compressed air or any other gas, added to the annulus, in case the diffusion rate of gas through the inner sheath of the flexible riser is too low to make frequent measurements. The exemplary embodiments also include the use of only one system to monitor multiple flexible pipes or risers.

Accordingly, in exemplary aspect of the present invention there is provided a method and system for monitoring a flexible pipe, including an inline sensor system coupled to the annulus of the flexible pipe to detect corrosion of the flexible pipe. The method and system may further comprise a pressure and flow measurement system coupled to the annulus of the flexible pipe for detecting water flooding of the pipe annulus. The pressure and flow measurement system includes a flow controller for controlling a flow of fluid through the system in a regulated or non-regulated manner.

In a further exemplary aspect of the present invention there is provided a method and system for monitoring an amount of water being accumulated in an annulus of a flexible pipe, including locating a pressure measurement system proximate to the annulus of the flexible pipe for measuring pressure of gas inside the annulus; controlling a flow of vent gas with a vent gas valve; positioning a flow measurement system upstream or downstream of the vent gas valve for measuring the flow of the vent gas when the vent gas valve is opened; and collecting with a microprocessor pressure and flow measurement data from the pressure measurement system and the flow measurement system and for determining the amount of water accumulated in the annulus based on the collected pressure and flow measurement data.

The exemplary aspects of the present invention can be practiced alone or in combination, as will be appreciated by those skilled in the relevant arts.

Still other aspects, features, and advantages of the present invention are readily apparent from the entire description thereof, including the figures, which illustrate a number of exemplary embodiments and aspects and implementations. The present invention is also capable of other and different embodiments and aspects, and its several details can be modified in various respects, all without departing from the spirit and scope of the present invention. Accordingly, the drawings and descriptions are to be regarded as illustrative in nature, and not as restrictive.

### BRIEF DESCRIPTION OF THE DRAWINGS

The embodiments of the present invention are illustrated by way of examples, and not by way of limitation, in the figures of the accompanying drawings and in which like reference numerals refer to similar elements and in which:

FIG. 1 illustrates an exemplary flexible pipe (or riser) structure;

FIG. 2 illustrates a high level diagram of exemplary inline system for monitoring of the flexible pipe structure of FIG. 1;

FIG. 3 illustrates an exemplary inline analyzer for the exemplary inline system of FIG. 2;

FIG. 4 illustrates an exemplary IR-based inline analyzer (e.g., an infrared spectrometer), for the exemplary inline system of FIG. 2;

FIG. 5 illustrates an exemplary valve-based inline analyzer for the exemplary inline system of FIG. 2;

FIG. 6 illustrates an exemplary microfluidic-based inline analyzer for the exemplary inline system of FIG. 2;

FIGS. 7A-7B illustrate a flexible pipe with dry annulus versus a flooded annulus;

FIG. 8 illustrates an exemplary system for determining flexible pipe annulus flooding;

FIG. 9 illustrates an exemplary system with regulated flow for determining flexible pipe annulus flooding;

FIG. 10 illustrates an exemplary system with non-regulated flow for determining flexible pipe annulus flooding; and

FIG. 11 illustrates a further exemplary system for determining flexible pipe annulus flooding for multiple risers.

### DETAILED DESCRIPTION

Various embodiments and aspects of the invention will now be described in detail with reference to the accompanying figures. Still other aspects, features, and advantages of the present invention are readily apparent from the entire description thereof, including the figures, which illustrates a number of exemplary embodiments and aspects and implementations. The invention is also capable of other and different embodiments and aspects, and its several details can be modified in various respects, all without departing from the spirit and scope of the present invention. Accordingly, the drawings and descriptions are to be regarded as illustrative in nature, and not as restrictive. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited. Likewise, the term “comprising” is considered synonymous with the terms “including” or “containing” for applicable legal purposes.

In this disclosure, whenever a composition, an element or a group of elements is preceded with the transitional phrase “comprising,” it is understood that we also contemplate the same composition, element or group of elements with transitional phrases “consisting essentially of,” “consisting,” “selected from the group of consisting of,” or “is” preceding the recitation of the composition, element or group of elements and vice versa.

All numerical values in this disclosure are understood as being modified by “about.” All singular forms of elements, such as armor wires, or any other components described herein including (without limitations) components of the flexible pipe or riser are understood to include plural forms thereof and vice versa.

In all embodiments of this invention a flexible pipe can be used. The term flexible pipe is known in the art, and it includes any suitable flexible structure that can be used in the invention, including, without limitation, flexible riser and flowlines.

### First Embodiment

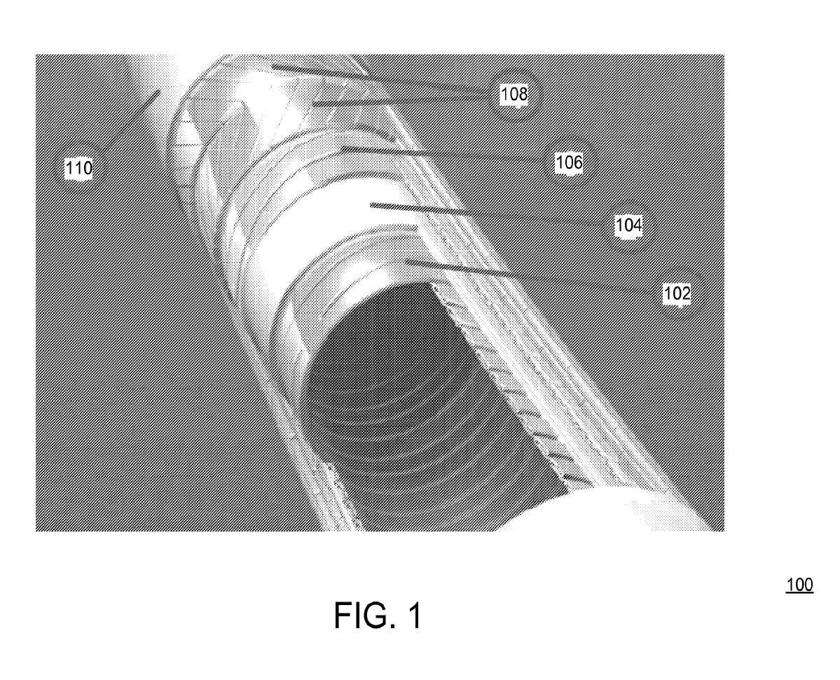

Referring now to the drawings, wherein like reference numerals designate identical or corresponding parts throughout the several views, and more particularly to FIG. 1 thereof, there is illustrated a flexible pipe structure 100 which includes a carcass layer 102 (which may also be referred to as “carcass 102”) that is designed to keep the pipe 100 from collapsing to the external pressure to which the pipe 100 is subjected and is in permanent contact with the produced fluid. The carcass layer 102 is usually manufactured from a corrosion resistant material. A pressure sheath 104 (which may also be referred to as “inner sheath 104” or “inner liner 104”) is a polymer layer that is provided and designed to act as a pressure barrier keeping the produced fluids in the flexible pipe 100. A failure of the pressure sheath 104, for example, due to cracks and fissures, and the like, might allow fluid to enter the annulus and although it is designed to be impermeable to fluid, with time gases might permeate through the pressure sheath and also gain access to the annulus. There are a multitude of aggressive gases that might be present in an oil and gas well, including gases, such as CO₂ and H₂S, which are corrosive to armor wire layers 108 and a pressure vault layer 106, which are normally manufactured from a “sweet” metal, for example, such as carbon steel, and the like. The armor wire layers are designed to keep the pipe 100 from bursting from the internal pressure of the produced fluid and they also support the weight of the pipe 100 itself. A failure of the armor wire layers could result in the rupture of the pipe 100, as the pressure vault layer 106 and the carcass layer 102 are not designed to withstand the weight of the flexible pipe 100. The armor wire layers 108 are protected from the outside fluid (e.g., sea water) by a layer 110 of polymer, usually called the outer sheath, and the void space between the outer sheath 110 and the pressure sheath 104 is called the annulus. Damage to the outer sheath 110 is one of the most common failure drivers seen in the industry and it could be caused by objects (e.g., tow boats, dragging anchors, dropped objects, etc.) impacting the flexible pipe 100, marine growth, friction with a loose object, among other reasons. The significance of monitoring the damage of the outer sheath 110 resides in the fact that the wire layers and the pressure vault layer are usually made out of a sweet metal that readily corrodes in contact with fluids, such as sea water, as mentioned above. Corrosion or corrosion-fatigue (e.g., fatigue induced or accelerated by corrosion) poses a potential danger to the integrity of the flexible pipe 100.

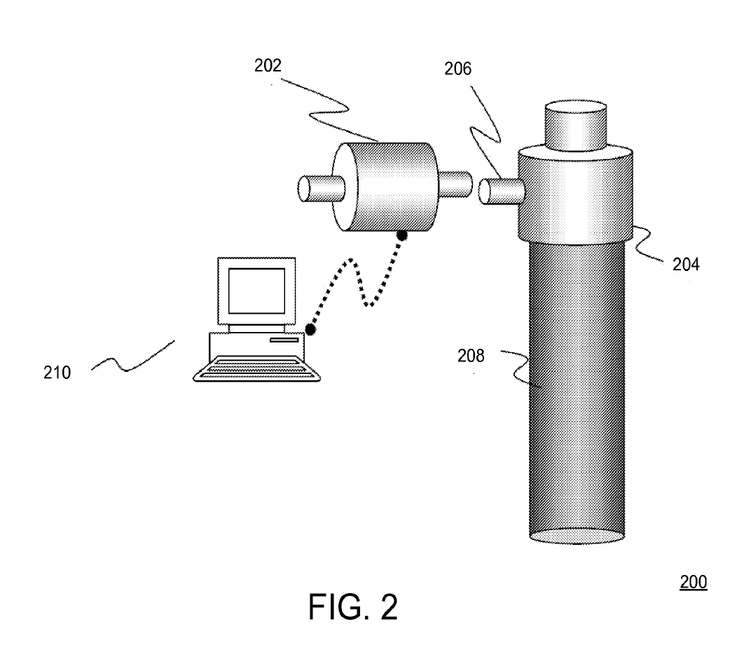

The annulus of a flexible pipe 208, as shown in FIG. 2, is open at the riser base 204 via a vent port 206 to allow venting gases which access the annulus by percolating from the producing fluid through the pressure sheath 104. If these gases are not vented, there is a risk of ballooning of the annulus that will result in the outer sheath 110 bursting. Although not commonly used in the industry, there are set procedures to monitor the chemistry of the gases produced from the annulus by taking samples and sending them to a laboratory to monitor aggressive/corrosive gases, for example, such as H2S, CO2, and the like. Gases generated from the chemical reaction of an actively corroding one or more armor wire layers and/or pressure vault layer can be identified by monitoring the presence of hydrogen fraction. Damage to the pressure sheath 104 can be inferred if gases from produced fluids, for example, such as methane or ethane, are identified in the sample. Water or water vapor is an indication that the outer sheath 110 could be damaged and the annulus is flooded with sea water.

As one or more of the armor wire layers and/or pressure vault layer made of iron corrodes, it frees hydrogen, as shown below. The following redox reaction occurs only in the presence of water and is crucial to the formation of rust:

2Fe²⁺+0.5O₂→2Fe³⁺+O²⁻

Additionally, the following multistep acid-base reactions affect the course of rust formation:

Fe²⁺+2H₂O⇄Fe(OH)₂+2H⁺

Fe³⁺+H₂O⇄2Fe(OH)₃+3H⁺

It is such hydrogen that with suitable equipment can be measured and monitored. There are several options in the industry of gas analyzers that can accurately measure the type and volume of the gases needed to monitor the integrity of flexible pipes, for example, including analyzers based on gas chromatography, mass spectrometry, (IR) spectroscopy, electrochemical sensors, catalytic sensors, microfluidic analyzers, tunable laser diode absorption spectroscopy, and the like.

Some of the reasons why the above method is not widely used are that the volume of gas produced out of the vent port 206 could be as low as 0.11/day, which makes sampling difficult, and the fact that sampling and sending such samples to a laboratory for analysis is time consuming and costly. Alternatively, the industry has also opted to monitor the volume of the gases produced from the vent port 206, wherein a change of volume will point to a possible failure of the pressure sheath 104, as fluids from the well enter the annulus. This method is only useful if the changes of volume are drastic in a relatively short period of time and the breach of integrity of the pressure sheath 104 is continuous in time. However, a failure of the outer sheath 110, corrosion of one or more of the armor wire layers and/or the pressure vault layer, and migration of aggressive/corrosive gases into the annulus, will most likely not be identified by such a volume monitoring method.

The two methods mentioned above suggested by the industry for monitoring the gases and volume of such gases produced from the annulus of a flexible pipe have several limitations that account for the lack of wide implementation and which advantageously are addressed by this exemplary embodiment. Historically the problems seen with these two methods are the impracticality to take, ship, and analyze samples of the gases produced in the vent port 206, the small volume of gas produced, and the little information that a change of volume of the produced gas by itself can entail.

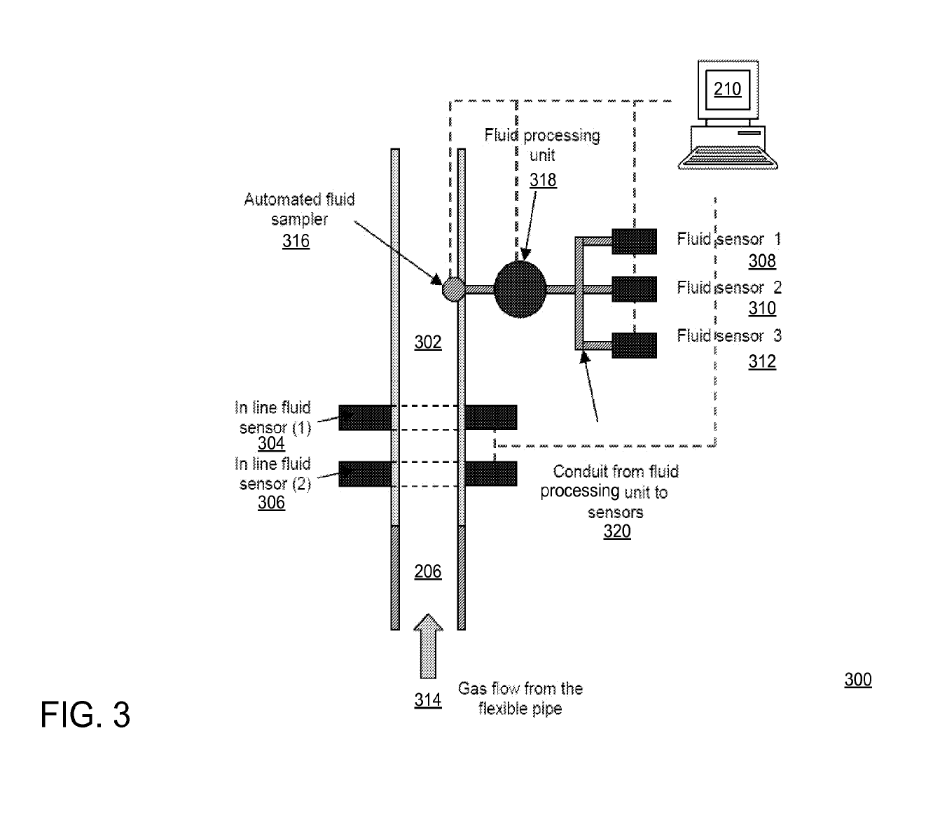

Accordingly, this disclosure addresses these limitations. In exemplary aspect, an inline analyzer 202 is coupled with a data recording and processing unit 210 (FIG. 3) (e.g., a personal computer, laptop computer, etc.), and software interface is used to monitor and record the type and volume of the produced gases. Suitable software that models the measurements of the gas type levels and volumes can be used to determine and report on the integrity of the pipe.

By measuring individual gas levels and the volumes produced over time by the inline analyzer 202 permanently fitted to the vent port 206, advantageously, it is possible, with suitable software, to infer if the integrity of the pressure sheath 104 has been compromised, if one or more of the armor wire layers and/or the pressure vault layer are actively corroding, if the annulus is flooded with sea water, if there are other failure drivers, and the like.

FIG. 3 illustrates an exemplary configuration 300 of the inline analyzer of FIG. 2. In FIG. 3, the analyzer 300 includes a flow line 302 connected to or part of the vent port 206 of a flexible pipe and one or more sensors 304-312 connected to the flow line 302. The sensors 304-312 can be configured differently to achieve real time monitoring. For example, the sensors 304-306 can be located in-line and coupled via conduit 320 to the data recording and processing unit 210 (e.g., a personal computer, laptop computer, etc.) analyze the flow of gas 314 flowing through the pipe without sampling the gasses.

In a further exemplary embodiment, an automated fluid sampler 316 is provided for providing samples to a fluid processing unit 318 coupled via conduit 320 to the one or more fluid sensors 308-312. The data recording and processing unit 210 (e.g., a personal computer, laptop computer, etc.) can control the automated fluid sampler 316, the fluid processing unit 318, and the one or more fluid sensors 308-312 to analyze the collected samples. The various sensors 308-312 can be associated with the active inline micro-sampler 316 that diverts part of the flowing gas 314 to the sensing system. The active inline sampler 316 is configured to automatically take samples, for example, according to a predefined periodic sequence (e.g., every second, minute, hour, day, week, month, year, etc.). This sequence can be a configurable function based on user need. The samples can then be processed through the processing unit 318 before being sent to the sensors 308-312. The processing performed by the processing unit 318 can include changing sample pressure, injecting a chemical into the sample, separating gases from the sample, or any other suitable processes employed to allow the sensors 308-312 to accurately measure sample properties. The processed fluid from the processing unit 318 is then directed via the conduit 320 to the sensing elements (i.e., sensors) 308-312.

The above exemplary configurations are very well suited for measurement techniques, such as near infrared (NIR) spectroscopy (e.g., based on absorption or reflection), and the like. They are also well suited for in-line chemical sensors, such as an H₂S sensor, as described in U.S. Pat. No. 6,939,717, a CO₂ sensor, as described in U.S. Pat. No. 6,995,360, a hydrocarbon analysis based sensor, as described in U.S. Pat. No. 4,994,671, all of which are incorporated by reference herein.

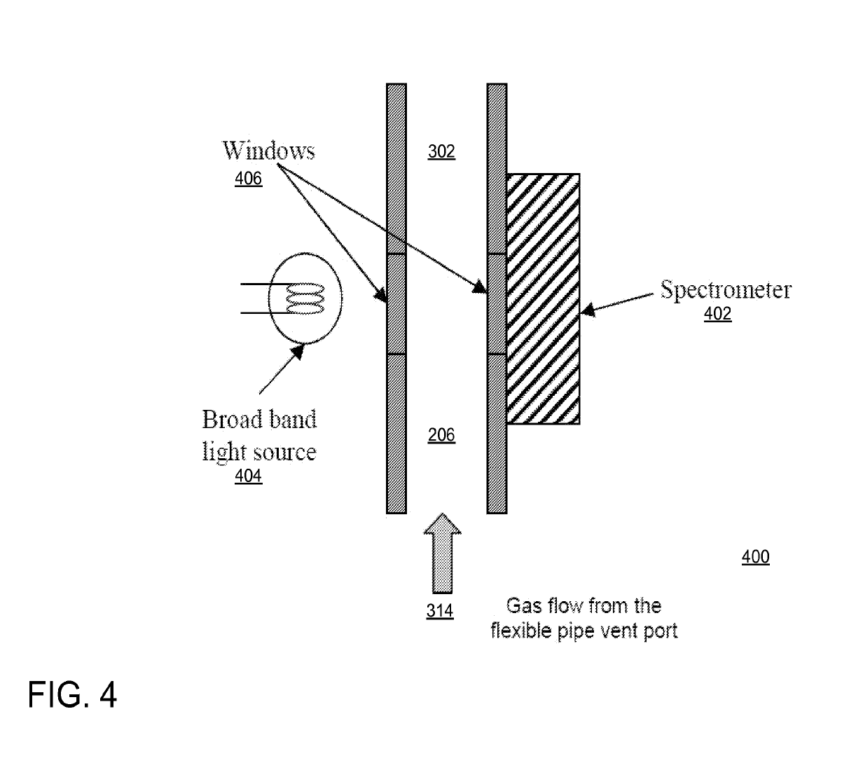

FIG. 4 illustrates an exemplary configuration 400 with a near infrared (NIR) spectrometer 402. This kind of configuration can be very useful for hydrocarbon and CO2 analysis. This configuration includes a broadband light source 404, and a pair of windows 406 connected to the flow line 302 and the spectrometer 402 tuned in the NIR region (e.g., as described in U.S. Patent Application No. 2007/0109537, incorporated by reference herein). This configuration is used to measure the optical absorption of the fluid at different wavelengths and then retrieve the fluid composition. The data recording and processing unit 210 (e.g., a personal computer, laptop computer, etc., not shown) can control the light source 404 and the spectrometer 402 to analyze the flow of gas 314 flowing through the pipe without sampling the gasses.

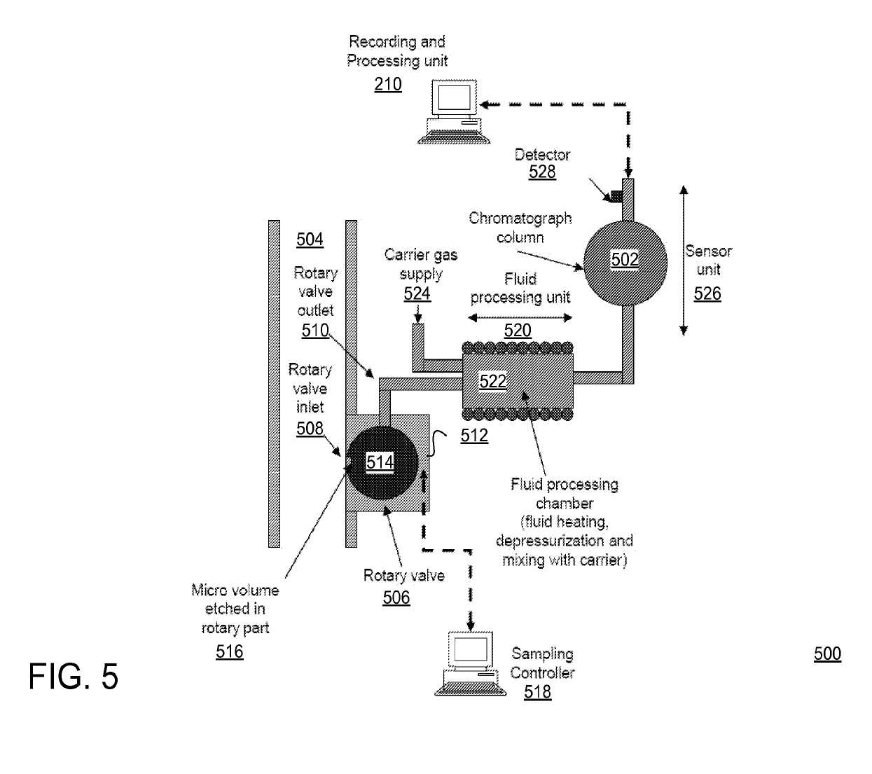

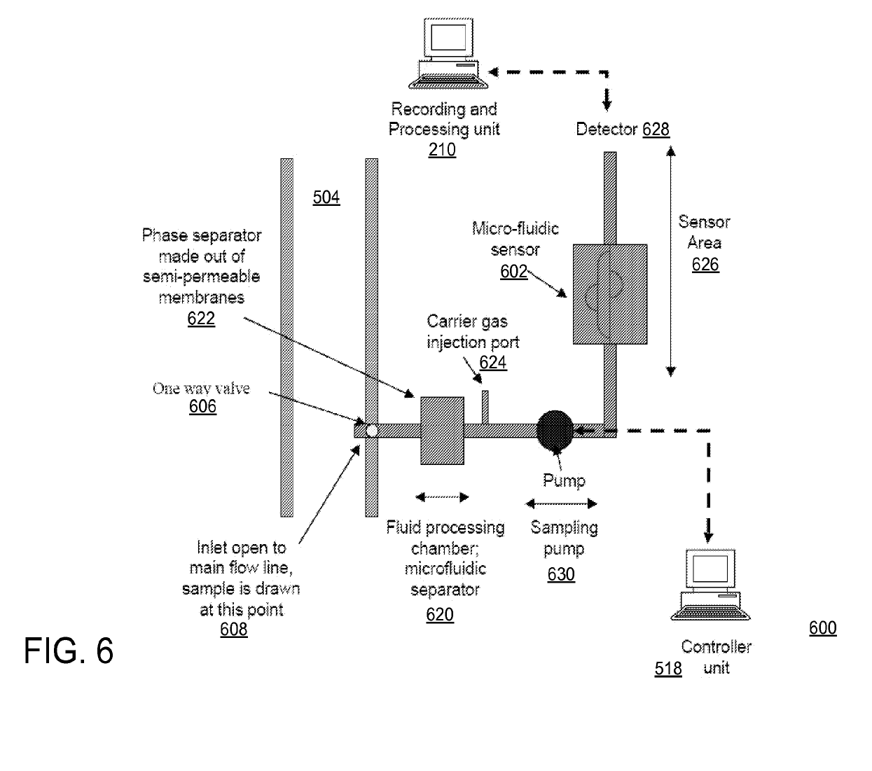

FIGS. 5 and 6 illustrate respective exemplary configurations 500 and 600 employing gas chromatography and a microfluidic device (e.g., as described in U.S. Patent Application No. 2006/0008913 or European Patent Application No. EP07291432.8, incorporated by reference herein). In FIG. 5, the configuration 500 includes a gas chromatograph 502 for the analysis of fluid from the vent port 206 (not shown). The sampler 500 further includes a sampling tube 504 connected to or as part of the main flow line 302 (not shown) and a rotary valve 506. The rotary valve 506 includes an inlet port 508, an exit port (or outlet) 510, a main body 512, an internal rotating part 514, and a micro-cavity 516 etched in the rotating part 514. The rotary valve 506 is coupled to a fluid processing unit 520 having a fluid processing chamber 522 and a carrier gas supply inlet 524. The fluid processing unit 520 is coupled to a sensor unit 526, including the gas chromatograph 502 coupled to a detector 528. The sensor unit 526 is coupled to the data recording and processing unit 210 (e.g., a personal computer, laptop computer, etc.).

The rotating part 514 movement is managed through a controller 518 (e.g., a processor, micro-controller, etc.). The automated sampling session is initiated, as follows. The controller 518 sends a signal to the rotary valve 506 to start the sampling process. The rotary valve 506 is rotated to align the micro-cavity 516 with the inlet port 508. The micro-cavity 516 fills up with a gas sample. The volume of the sample for this type of analyzer is in the range of a few micro-liters. The rotary valve 506 rotates so that the micro-cavity 516 position is in front of the exit port 510. Then, the sample is injected into the processing chamber 522.

In the chamber 522, the gas pressure and temperature are adjusted. Usually, the gas chromatograph 502 operates at high temperature and requires a pre-heating of the sample to make sure that all the components of the sample are in the gas phase. The sample gas pressure can be reduced to atmospheric pressure, while the temperature can be increased to above 150.degree. C., wherein the final temperature depends on the type of species of sample gas injected, and the type of the gas chromatograph column 502 employed. Then, the sample is mixed with a carrier gas (e.g., N2, etc.) via the carrier gas supply inlet 524 and injected in the sensor section 526.

As noted above, the sensor section 526 includes the gas chromatograph column 502 and the detector 528 located at the exit of the column 502. The different components of the injected gas travel at different speeds through the column 502. The detector 528 (e.g., a flame ionization detector, etc.) then detects the different components as they arrive at the top of the column 502. The detector 528 is used to estimate the time it takes for the different components to travel through the column 502. The time of travel through the column 502 is directly linked to the nature of the chemical compound. Advantageously, gas chromatography allows the detailed analysis of a complex chemical mixture, and is very well suited for the analysis of hydrocarbon compounds, CO2, H2, H2S, and the like.

In FIG. 6, the exemplary configuration 600 includes a microfluidic device 602, as a sensing element. The microfluidic device 602 can be used for chemical sensing, for example, as described in U.S. Patent Application No. 2006/0008913, incorporated by reference herein. The terms “microfluidic system” or “microfluidic device” can include a network of one or more channels with dimensions of tens to hundreds of micrometers that can have one or more components, for example, including pumps, valves, mixers, integrated optical fibers, and other suitable components integrated on a chip for the purpose of manipulating and/or analyzing minute amounts of fluids, and the like. The term “fluid” can include a continuous, amorphous substance whose molecules move freely past one another and that has the tendency to assume the shape of its container, including both liquids and gases, and the like.

In an exemplary aspect of this embodiment, the sampling is done through a sampling tube 504 connected to or as part of the main flow line 302 (not shown) and used in conjunction with a sampling pump 630. The pump 630 is controlled by an external controller 518 (e.g., a processor, micro-controller, etc.), which sets a predetermined periodic activation sequence. The sample proceeds through a phase separation membrane 622 in a microfluidic phase separator 620. This separator ensures that only gas will reach the microfluidic device (or sensor) 602. The microfluidic device 602 can include an integrated micro-gas chromatograph (not shown), which can perform the analysis of hydrocarbon compounds, CO2, H2, H2S, and the like. Microfluidic based gas chromatographs have the advantage of reduced carrier gas consumption as well as smaller size. As shown in FIG. 6, the exemplary configuration 600 further includes a one way valve 606, inlet port 608, carrier gas injection port 624, and sensor area 626 including the microfluidic device 602 and a detector 628 coupled to the data recording and processing unit 210 (e.g., a personal computer, laptop computer, etc., not shown).

In FIGS. 2-6, the data recording and processing unit 210 can be configured to perform systematic permanent monitoring and analysis of gases in the flexible pipe structure 100 of FIG. 1 via one or more of the in-line sensors coupled with a software interface that records the level of produced gas types and alerts the user if such levels are above the normal limit. The software can be configured to actively monitor gases resulting from the chemical reactions, e.g., hydrogen, for example, when the metal of the armor wires and/or the pressure vault layer of a flexible pipe, riser, or other structure, corrodes. Changes of the flowrate of produced gas can be inferred as degradation of the pressure sheath that allows gas and fluid from the production fluid to fill the annulus, and presence of water vapor indicating an annulus flooded with sea water. The software interface can monitor and record the levels of produced gases. Advantageously, the level of produced gases can be analyzed with the software to identify if the integrity of the pressure sheath has been compromised, whether there is sea water entry in the annulus, whether one or more of the armor wire layers and/or the pressure vault layer is corroding, whether the outer sheath has been damaged, other failure drivers, and the like.

### Second Embodiment

The present invention includes recognition that flexible pipes or risers have some drawbacks. Referring again to FIG. 1, the typical flexible pipe or riser structure 100 includes the many layers, each of which plays a different role from providing structural strength to providing isolation between the inside bore, which carries producing fluids, from the outside sea water. The steel reinforcing layers (armors 108 and pressure vault layer 106) are contained within a very confined environment called the annulus which is located between the inner polymer sheath 104 and the external polymer sheath 110. The inner sheath 104 is the barrier to the conveyed production fluids and the external sheath 110 protects against the seawater environment. If this annulus has water present in it, then the longer term integrity of the flexible riser 100 is compromised because of corrosion. It should be noted that although the inner 104 and outer sheaths 110 are impermeable, under high temperature and pressure conditions small amounts of gases can permeate through the inner sheath 104. Corrosive gases are often present in production fluids (e.g., H₂S, CO₂ and water vapour), and hydrocarbons, such as CH₄, and can diffuse through the inner sheath 104 and accumulate in the annular space. This results in a corrosive environment in contact with the steel members 106 and 108 (which may be and usually are made of carbon steel), which can significantly reduce the life of the flexible pipe 100.

In addition, it is often the case that the outer sheath 110 is damaged (e.g., possibly only lightly damaged) during installation, which can cause a very slow ingress of seawater over time. Sometimes the outer sheath 110 can be seriously breached resulting in flooding of the annulus. Very slow diffusion of water through the outer sheath 110 is also possible. In such cases, water enters the annulus causing corrosion of the steel wire structures 106 and 108 (i.e., the vault layer 106 and armor wire layers 108, respectively), which can result in premature failure of the flexible pipe 100.

Clearly the failure of a flexible riser 100 is very costly and can result in catastrophic damage to the environment. If, however, the failure is detected early and monitored, advantageously, repair or replacement can be scheduled in order to significantly reduce the risk of environmental damage and minimize production down-time.

Determination of possible water in the annulus, according to current approaches, includes periodically monitoring the vented gas from the annulus and comparing the monitoring data with complex theoretical diffusion values. However, such approach is not very accurate and small amounts of water intrusion are very difficult to detect. In addition, with such approach, it is not possible to discriminate between an increase in gas diffusion rate and a slow leak of seawater into the riser annulus. Another approach involves periodically pulling a vacuum at the surface on the vent lines that connect to the annulus. The degree to which a vacuum can be held is used to give an indication of a leak in the inner sheath 104 or the outer sheath 110. In practice, however, such a method is recognized as slow, expensive, difficult to control and not very reliable. Accordingly, it is not practical to perform frequently such a vacuum test.

In an exemplary aspect of this embodiment there is provided a system and method for monitoring the amount of water being accumulated in the annulus of a flexible pipe, the system including a pressure measurement system located proximate to the annulus for measuring the pressure of the gas inside said annulus, a vent gas valve for controlling the flow of the vent gas, a flow measurement system positioned upstream or downstream of the vent gas valve for measuring the flow of the vent gas when the vent gas valve is opened, a microprocessor for collecting the pressure and flow measurement data and providing the amount of water accumulated in the annulus. In operation, the vent gas valve is typically closed, in which case the pressure of the vent gas of the annulus will be increasing over time, as vent gas and/or water will be accumulating in the annulus. The pressure measurement system monitors the pressure of the vent gas and when the pressure reaches a certain value, then the vent gas valve can be opened and vent gas can flow through the flow measurement system. By monitoring the pressure change with time and the flow rate of the vent gas with time, such data can be used to calculate the volume of the gas in the annulus with increased accuracy and from such calculation the amount of water that has been accumulated in the annulus can be determined. The process can be repeated at predetermined time intervals. In addition, the pressure buildup of the vent gas in the annulus that occurs when the vent gas valve is closed can provide useful data regarding the amount of water in the annulus.

The exemplary system and method advantageously provide for continuous monitoring of the pressure of the vent gas in the annulus and the flow rate of the vent gas in the vent line. In an exemplary embodiment, a temperature sensor is positioned in the annulus for measuring the temperature of the vent gas in the annulus and allowing to take into account the effect of temperature changes in the estimation of the amount of water in the annulus.

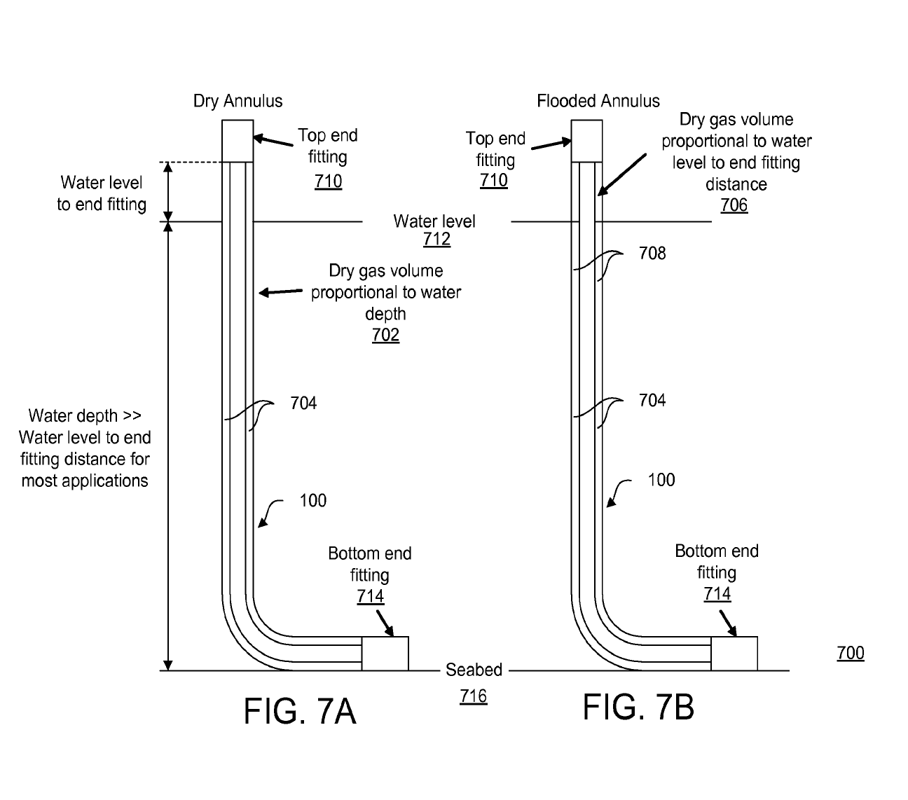

Accordingly, the further exemplary aspects of this embodiment, to be described with respect to FIGS. 7-11, address the above and other problems with the current approaches, by providing a novel system that measures the annular pressure and vented gas flow rate to determine not only the annulus volume (and thus the liquid level), but also the diffusion rate during production. The determined information, advantageously, can be used to validate design models, as well as serving as a real time alert system for detecting liquid loading of the annular space. The time progression of the liquid column in the annulus further serves to identify the liquid present as condensation or sea water invasion, since the liquid condensation, as part of the diffusion process through the pressure sheath 104 is significantly slower than a breach of the outer sheath 110 that could cause sea water invasion. Advantageously, the exemplary aspects of FIGS. 7-11 can be combined with any and all aspects of the exemplary embodiment of FIGS. 1-6 to provide a novel annulus monitoring system, including pressure and flow measurement for detecting annulus flooding, along with hydrogen sensing to detect ongoing corrosion.

Accordingly, FIGS. 7A-7B illustrate an exemplary system 700, including a flexible pipe 100 with a dry annulus 704, as shown in FIG. 7A, versus a flooded annulus 704, as shown in FIG. 7B. In FIG. 7A, in the dry condition, the fluid 702 inside the annulus 704 is mostly gas resulting from the permeation of gas in the bore to the annulus 704 through the inner sheath 104 (not shown) (which may also be referred to herein as “inner liner 104”) of the flexible pipe 100. In FIG. 7B, in the flooded condition, the volume of gas 706 is significantly reduced as the water 708 has invaded the annulus 704. As shown in FIGS. 7A-7B, the exemplary system 700 further includes a top end fitting 710 above the water level and a bottom end fitting 714 on the seabed 716.

In the context of FIGS. 7A-7B, in an exemplary aspect of this embodiment, the dry or flooded condition of the annulus 704 is assessed by monitoring the mass of free gas accumulating in the annulus 704. An exemplary method includes monitoring the accumulating pressure in the annulus 704. If the diffusion rate of gas through the inner sheath 104 is not sufficient, the riser annulus 704 can be pressured up, for example, with compressed air or any other suitable gas. Next, a controlled release of the accumulated gas is performed and the gas pressure and mass or volume flow rate is monitored over time during the release. The volume and/or the mass of gas released during the release between two annulus pressure conditions is then monitored and used to estimate the dry volume of gas in the annulus 704. Additionally, the pressure build up between controlled releases can be monitored and used to estimate diffusion rates.

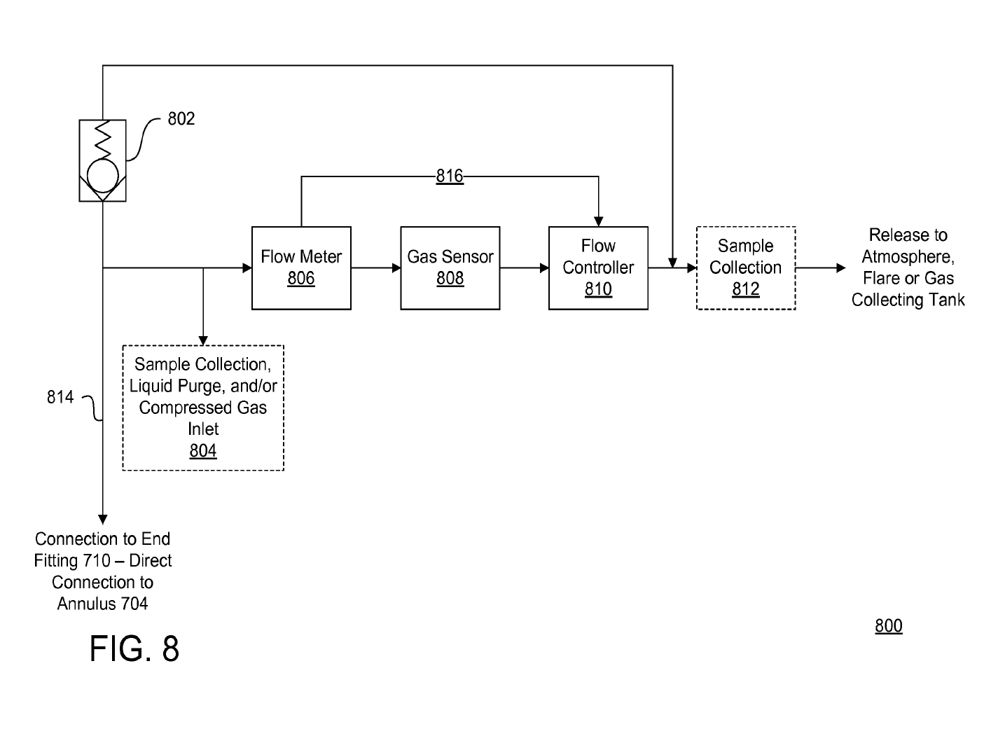

FIG. 8 illustrates an exemplary system 800 for determining the flooding of the annulus 704 of the flexible pipe 100. In FIG. 8, the exemplary system 800 includes a flow controller 810 for controlling the flow through the system in either a regulated or non-regulated fashion. The most straightforward flow controller 810 employs a variable restriction, such as a valve that can be electrically controlled, and the like. The flow through such a valve can be regulated, for example, based on a feedback-loop 816 based on a flow meter section 806 reading. Flow regulation also can be achieved with a passive device, for example, including a sonic nozzle having a restriction designed such that the gas flow accelerates to a critical velocity equal to the local sonic velocity at the nozzle throat. Therefore, the gas velocity is forced to sonic velocity, leading to a known and constant volumetric flow rate.

The exemplary system 800 further includes a gas sensor 808 section (also referred to herein as “fluid sensor section”) for measuring the gas pressure of the annulus 704 in any of known manners. The gas sensor section 808 can be retrofitted with physical or chemical sensors for the detection of components that have critical importance to the riser 100 integrity, such as H2S and CO2, or to improve either the flow measurement or the interpretation, such as sensors for gas density, temperature, sound velocity, and the like. A sensor can also be added to detect by-products of corrosion processes in the armor wires 108 or the pressure vault 106.

The exemplary system 800 further can include an optional sample collection port 812 to enable collection of vented gas for lab analysis. Depending on location of the sample collection port 812 with respect to the flow controller 810, the flow controller 810 can enable the sample collection 812 at particular times, for example, as determined by a user or as automatically programmed into the system 800 either on a periodic basis or triggered by a condition of the annulus 704 changing as detected by the available sensors 808. In FIG. 8 two possible locations for the optional sample collection port are shown, 804 and 812. Other optional functions 804 that can be implemented on such a port include a water or liquid purge, or compressed gas inlet. As shown in FIG. 8, the vented gas can be released into the atmosphere, to a flare for burning, or a gas collecting tank.

Depending of the configuration, the system 800 can include the volumetric or mass flow meter 806. For example, the use of the flow meter 806 may be required when the flow is not regulated. The volumetric/mass flow meter 806 can measure the volume/mass flowrate of gas flowing through the system connected to the pipe 100 end fitting 710 gas outlet or the annulus 704 via connection 814. Several types of meter technologies can be used for the flow meter 806. For example, for volumetric metering, possible implementations include a positive displacement meter (e.g., a piston meter), a rotary meter, an ultrasonic meter, and the like. A mass flow meter implementation can be based on a thermal flow meter, a Coriolis meter, and the like. Further possible implementation can include the combination of a volumetric meter with a gas pressure or density sensor, and the like.

Advantageously, the exemplary aspects of the embodiment can by-pass a safety check valve 802 of the pipe 100. The check valves 802 are used on the end-fitting 710 and are spring loaded valves, although any other suitable valves can be used. The flow generated by the valve 802 during the release phase is not very well controlled. Accordingly, the exemplary measuring system can be connected directly to the annulus 704, as discussed above, or to the end fitting 710, while bypassing the valve 802.

Monitoring of the pressure during the pressure build-up is also advantageous, wherein the pressure change rate over time dp/dt is proportional to dMgas/dt, and the produced gas will mostly come from the diffusion from the production bore to the annulus 704 through the inner polymer sheath 104. Therefore, the monitoring of dp/dt can be used to monitor the evolution of the diffusion rate with time as well as possible damage of the inner sheath 104. As shown in FIG. 8, the analyzed vent gas can be either released to the atmosphere, sent to a flare to be burned, or stored in a containing volume (e.g., a gas collecting tank) that would allow disposal later on in a controlled environment.

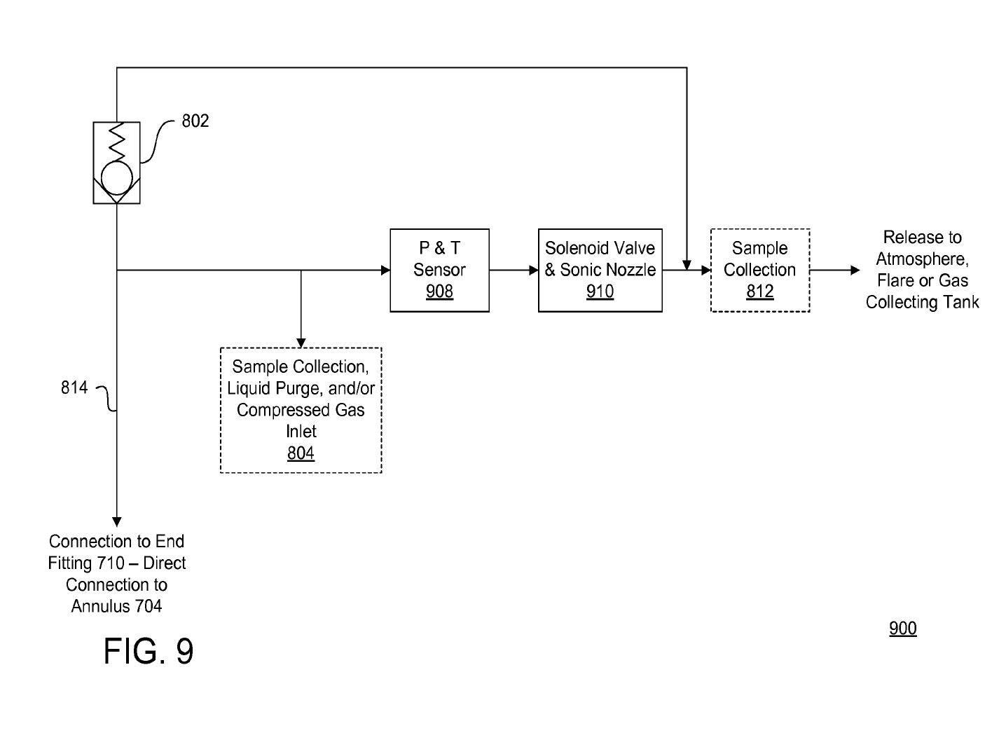

FIG. 9 illustrates another exemplary system 900 with regulated flow for determining flexible pipe annulus flooding. The common elements of the exemplary system 900 with the exemplary system 800 of FIG. 8 will not be further described for the sake of brevity. In FIG. 9, the exemplary system 900 includes a pressure and temperature sensor 908, a flow controller 910 that includes a solenoid valve and a sonic nozzle. The solenoid valve is used to open or close the flow, but not regulate the flow, whereas the flow is regulated to a given flow rate by the sonic nozzle.

In an exemplary aspect of this embodiment, a measurement sequence can include accumulating the gas in the annulus 704. Then the solenoid valve is closed. As explained before, due to the diffusion process through the inner liner 104, gas accumulates in the pipe 100. The typical maximal acceptable pressure in the annulus 704 is about 3 bars. The safety check valve 802 can be set with an opening pressure around such a value. The pressure in the annulus 704 then is measured over time during the build-up with the sensor 908. Once the pressure reaches a preset value pMax below the check valve 802 opening value, a master controller (not shown) can be used to open the solenoid valve. Alternatively, compressed air or another gas can be introduced into the riser annulus 704 from the topside via the compressed gas inlet 804, as previously described. Fluctuations in the temperature of the annulus 704 will directly cause proportional changes in the pressure, wherein a continuous pressure measurement of high resolution will give information on the temperature fluctuations of the annulus 704. Next, a controlled release of the accumulated gas is performed, and the gas pressure is monitored over time to estimate the dry volume in the annulus 704.

Once the solenoid valve is open at time tstart, the gas starts flowing out of the annulus 704 and through the sonic nozzle. The sonic nozzle sets the volumetric flow rate Qset to a fixed value. The gas pressure pgas(t) and temperature Tgas(t) are recorded during the release.

The check valve 802 is closed once the pressure reaches a certain pre-set value pmin at time tend. It is then possible to estimate the total mass of gas M_{gas} that escaped from the annulus 702, as follows:

$${M}_{gas} = {\int }_{{t}_{start}}^{{t}_{end}} {Q}_{set} ⋆ {ρ}_{gas} ( {p}_{gas} ( t ) , {T}_{gas} ( t ) ) ⋆ dt = {Q}_{set} ⋆ {\int }_{{t}_{start}}^{{t}_{end}} {ρ}_{gas} ( {p}_{gas} ( t ) , {T}_{gas} ( t ) ) ⋆ dt$$

where p_{gas}(t) and T_{gas}(t) are the gas pressure and temperature measured during the release, and ρ_{gas}(p_{gas}(t),T_{gas}(t)) is the gas density estimated from pressure and temperature (T) measurements.

It can be noted that the expected gas in the annulus 704 is mostly methane (e.g., at more than 95%). Therefore, the gas density can be easily estimated from pressure and temperature measurements. If the composition were to change significantly over time, a direct measurement of the gas density can also be performed, as previously described.

From the mass of gas that escaped the annulus 704 and the corresponding pressure drop, one can use the law of perfect gas (e.g., a good approximation for methane at low pressure and moderate temperature) to compute the ratio of the volume of the annulus 704 to the absolute temperature. The dry volume then can be calculated, assuming the temperature of the annulus 704 is known. Any decrease in annulus temperature can also be estimated, assuming the dry volume cannot increase, wherein such a decrease can cause the formation of hydrates, or wax or asphaltene deposits, and reduce the flow capacity of the riser 100 and which can advantageously be detected by the exemplary system 900.

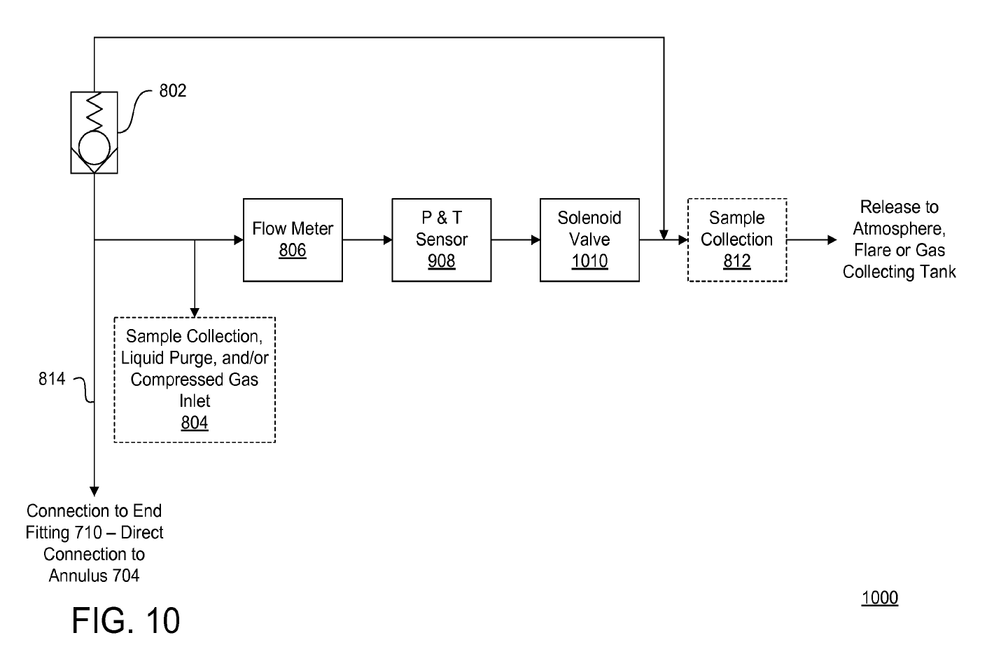

FIG. 10 illustrates an exemplary system 1000 with non-regulated flow and flow meter section for determining flexible pipe annulus flooding. The common elements of the exemplary system 1000 with the exemplary systems 800 and 900 of FIGS. 8 and 9, respectively, will not be further described for the sake of brevity. In FIG. 10, the exemplary system 1000 includes a volumetric or mass flow meter 806, a pressure and temperature sensor 908, a solenoid valve 1010 used to control the flow (e.g., having on/off positions). The volumetric gas flow meter 806 is used for measuring gas volumetric flow rate Q_{gas}(t).

The measurement sequence is the same as described above with respect to FIG. 9, wherein Q_{gas}(t), p_{gas}(t) and T_{gas}(t) are recorded as a function of time during the release phase. The mass of gas released M_{gas} can be estimated, as follows:

$${M}_{gas} = {\int }_{{t}_{start}}^{{t}_{end}} {Q}_{set} ( t ) ⋆ {ρ}_{gas} ( {p}_{gas} ( t ) , {T}_{gas} ( t ) ) ⋆ dt$$

The dry volume or temperature variation in the annulus 704 can then be estimated from M_{gas}, as previously described. As previously described, the exemplary aspects include the ability to take a sample of the vent gas via the sample collection ports 812 or 804. Advantageously, gas sampling can be used for performing analysis of the composition of the vented gas, for example, to allow the estimation of the corrosive species concentration, such as H₂S or CO₂ concentrations and their evolution over time.

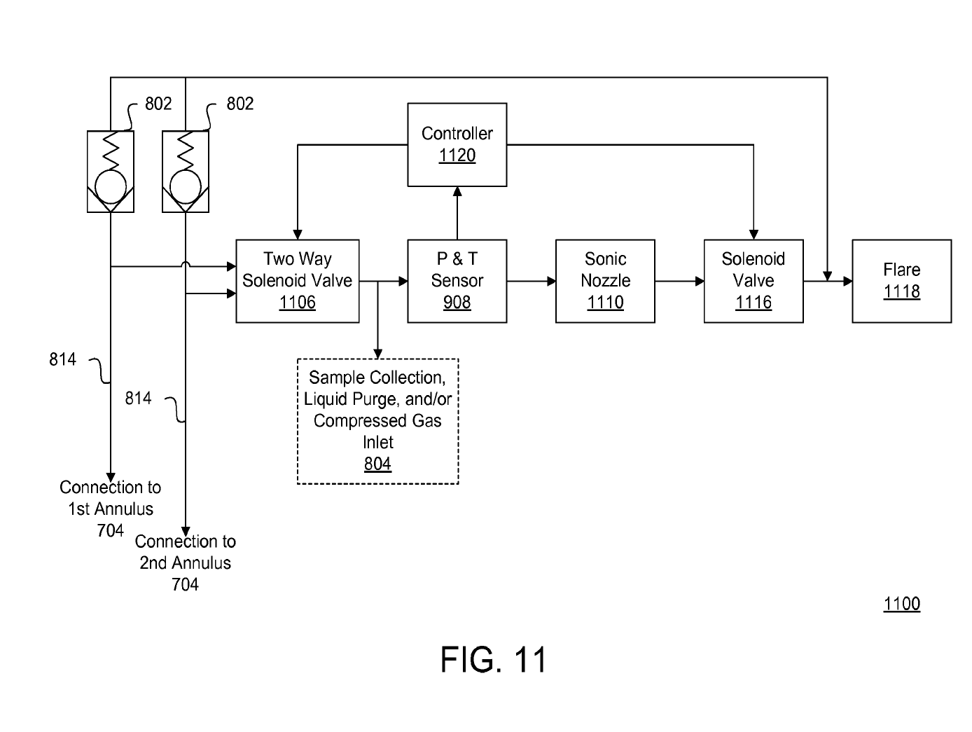

FIG. 11 illustrates an exemplary system 1100 for determining flexible pipe annulus flooding for multiple risers. The common elements of the exemplary system 1100 with the exemplary systems 800, 900 and 1000 of FIGS. 8, 9 and 10, respectively, will not be further described for the sake of brevity. In FIG. 11, each riser 100 employs a safety check valve 802 that is coupled to a flare 1118, but otherwise the sensors 908, flow controller and flow meter sections 1110 (sonic nozzle) and 1116 (solenoid valve), respectively, can be shared or multiplexed between multiple risers, by selecting one of multiple solenoid valve 1106 positions via a controller 1120. The time required for each vent operation is small compared to the time needed to accumulate pressure in the respective annulus 704, so the vent operations can be performed sequentially. The sequence of pressure measurement for each riser in turn, and of vent, when needed, can be performed automatically under computer control, with a sequence driven by the pressure measurement.

All or a portion of the devices and subsystems of the exemplary embodiments and all aspects thereof can be conveniently implemented by the preparation of application-specific integrated circuits or by interconnecting an appropriate network of conventional component circuits, as will be appreciated by those skilled in the electrical art(s). Thus, the exemplary embodiments, including their aspects, are not limited to any specific combination of hardware circuitry and/or software. In addition, one or more general purpose computer systems, microprocessors, digital signal processors, microcontrollers, and the like, can be employed and programmed according to the teachings of the exemplary embodiments (and aspects thereof) of the present inventions, as will be appreciated by those skilled in the computer and software arts. Appropriate software can be readily prepared by programmers of ordinary skill based on the teachings of the exemplary embodiments, as will be appreciated by those skilled in the software art(s).

All documents described or mentioned herein are incorporated by reference herein in their entirety, including any priority documents and/or testing procedures to the extent they are not inconsistent with this specification and for all jurisdictions in which such incorporation is permitted. As is apparent from the foregoing general description and the specific embodiments and aspects, while forms of the disclosure have been illustrated and described, various modifications can be made without departing from the spirit and scope thereof. Accordingly, it is not intended that the disclosure be limited thereby.

While the present inventions have been described in connection with a number of exemplary embodiments and aspects, and implementations, the present inventions are not so limited, but rather cover various modifications, and equivalent arrangements, which fall within the purview of the appended claims.

1. A system for monitoring corrosion in a flexible pipe having an annulus between inner and outer layers of the pipe with fluid and an armor wire therein and a vent port in fluid communication with the annulus, the system comprising:
an inline analyzer comprising:
a flow line connected to the vent port such that the fluid venting from the vent port flows into the flow line; and
a fluid sensor that detects the type of the fluid in the flow line; and
a data recording and processing unit coupled with the inline analyzer and comprising software to monitor and record the type of fluid in the flow line from the vent port based on communication with the in-line fluid sensor and detect corrosion of the armor wire by identifying a byproduct of the corrosion of the armor wire.

2. The system of claim 1, wherein the fluid sensor comprises a fluid analyzer based on one of a gas chromatograph, a mass spectrometer, infrared spectroscopy, an electrochemical sensor, a catalytic sensor, a microfluidic analyzer, and tunable laser diode absorption spectroscopy.

3. The system of claim 2, wherein the fluid sensor detects the type of fluid flowing through the flow line without physical removal of a sample from the flow line.

4. The system of claim 1, wherein the detection of the e of fluid flowing through the flow line is made in-situ.

5. The system of claim 1, wherein the detection of the e of fluid flowing through the flow line is performed in real time.

6. The system of claim 22, wherein the sampling of the annulus fluid flowing through the flow line is performed in-situ.

7. The system of claim 22, wherein the sampling of the annulus fluid flowing through the flow line is performed in real time.

8. The system of claim 1, wherein the inline analyzer and data recording and processing unit are configured to perform gas analysis including measuring a volumetric flow rate of a gas stream flowing from the vent port and fluid properties of the gas stream.

9. The system of claim 1, further comprising a pressure and flow measurement system coupled to the annulus of the flexible pipe for detecting water flooding of the pipe annulus, wherein the pressure and flow measurement system comprises: a flow controller for controlling a flow of fluid through the system in a regulated or non-regulated manner.

10. The system of claim 9, wherein the flow controller comprises a variable restriction.

11. The system of claim 10, wherein the variable restriction is an electrically controlled valve and the flow through the valve is regulated based on a feedback-loop based on a flow meter section reading.

12. The system of claim 9, wherein the flow controller comprises a passive device, including a sonic nozzle having a restriction configured such that fluid flow is accelerated to a critical velocity equal to a local sonic velocity at a throat of the nozzle and leading to a known and constant volumetric flow rate.

13. The system of claim 9, the pressure and flow measurement system further comprises a gas sensor section for measuring gas pressure of the annulus.

14. The system of claim 13, wherein the gas sensor section is configured to be retrofitted with physical or chemical sensors for detection of fluid components, including H2S and CO2, or to improve flow measurement or interpretation, including sensors for gas density, temperature, and sound velocity.

15. The system of claim 9, wherein the pressure and flow measurement system further comprises a sample collection port to enable collection of vented gas for laboratory analysis.

16. The system of claim 15, wherein the flow controller enables sample collection at time intervals determined by a user or as automatically programmed into the system either on a periodic basis or triggered by a condition of the annulus changing as detected by a sensor.

17. The system of claim 9, wherein the pressure and flow measurement system further comprises a volumetric or mass flow meter section to measure volume/mass flowrate of gas flowing through the system.

18. The system of claim 17, wherein the flow meter section comprises the volumetric flow meter section which includes a rotary meter, an ultrasonic meter, or a positive displacement meter comprising a piston meter.

19. The system of claim 17, wherein the flow meter section comprises the mass flow meter section which includes a thermal flow meter, or a Coriolis meter.

20. The system of claim 17, wherein the flow meter section comprises a combination of a volumetric meter with a gas pressure or density sensor.

21. The system of claim 9, wherein the system comprises more than one pressure and flow measurement systems coupled to an annulus of respective more than one flexible pipes for detecting water flooding of the annulus of the respective more than one flexible pipes.

22. The system of claim 1, wherein the inline analyzer further comprises a fluid sampler in fluid communication with the flow line that draws a sample from the flow line of the annulus fluid and another fluid sensor that measures a property of the sample.

23. A method for monitoring corrosion in a flexible pipe of a subsea installation the flexible pipe having an annulus between inner and outer layers of the pipe with fluid and an armor wire therein and a vent port in fluid communication with the annulus, the method comprising:
venting a gas stream from the annulus through the vent port into a flow line connected with the vent port;
monitoring the vented gas stream in real time via an inline fluid analyzer comprising a fluid sensor to detect the type of gas in the flow line;
using a data recording and processing unit to obtain data from the inline fluid analyzer related to the type of gas identify the gas in the flow line, and detect corrosion of the armor wire based on the gas identified as a byproduct of the corrosion of the armor wire.

24. The method of claim 23, further comprising determining a flow rate for the vent gas stream, comparing the determined flow rate to a base flow rate, and determining the integrity of the armor wire in the annulus of the flexible pipe from the gas analysis.

25. The method of claim 23, further comprising determining fluid properties of the vent gas stream.

26. The method of claim 23, further comprising:
taking a sample via a sampling mechanism of the inline fluid analyzer, the sampling mechanism including a sampling tube connected to the flow line and a rotary valve, the rotary valve including:
an inlet port;
an exit port;
a main body;
an internal rotating part; and
a micro-cavity etched in the rotating part; and
sending a signal from a controller to the sampling mechanism to start a sampling sequence, including:
rotating the rotary valve to align the micro-cavity with the inlet port so that the micro-cavity fills up with a fluid sample from the vent port of the flexible pipe;
rotating the rotary valve to align the micro-cavity with the exit port so that the sampled fluid exits through the exit port into a processing chamber;
injecting the sampled fluid into the processing chamber;
adjusting the pressure of the sampled fluid to atmospheric pressure and increasing the temperature of the sampled fluid to above 150° C.;
mixing the adjusted sampled fluid with a carrier gas and injecting the mixed sampled fluid into the sensor section of the fluid analyzer;
relaying via the fluid analyzer fluid analysis measurements of the mixed sampled fluid to a recording and processing unit;
monitoring and modeling via the recording and processing unit different types of fluids measured by the inline fluid analyzer and determining the integrity of the flexible pipe based thereon; and
automating the sampling sequence to recur at predetermined time intervals.

27. The method of claim 26, wherein the fluid analyzer comprises a gas chromatograph.

28. The method of claim 23, further comprising:
taking a sample from the inline fluid analyzer located at an exit of the vent port of the flexible pipe, the fluid analyzer having a sensor section for analysis of fluids and a sampling mechanism, the sampling mechanism including a sampling tube connected to a main flow line and a sampling pump activated by a controller;
sending a signal from the controller to the sampling mechanism to start a sampling sequence, including:
drawing out via the pump of the sampling mechanism a fluid sample from the main flow line into a cavity of the sampling mechanism;
injecting the fluid sample into a processing chamber;
passing the injected fluid sample through a series of phase separation membranes of a microfluidic separator;
mixing the passed fluid sample with a carrier gas and injecting the mixed fluid sample into the sensor section of the fluid analyzer;
relaying via the fluid analyzer fluid analysis measurements of the mixed sampled fluid to a recording and processing unit;
monitoring and modeling via the recording and processing unit different types of fluids measured by the fluid analyzer and determining the integrity of the flexible pipe based thereon; and
automating the sampling sequence to recur at predetermined time intervals.

29. The method of claim 28, wherein the fluid analyzer comprises a microfluidic analyzer.

30. The method of claim 28, wherein the fluid analyzer comprises a gas chromatograph.

31. The method of claim 23, further comprising:
taking a measurement from a system located at an exit of the vent port of the flexible pipe, the system including an in-line sensor mechanism for the analysis of fluids in the flow line, the in-line sensor mechanism including a light source coupled to a window in one side of a flow line, a second window located at opposite side of the flow line, and a spectrometer coupled to the second window, wherein the measurement is activated by a controller;
sending a signal from the controller to the in-line sensor mechanism to start a measuring sequence, including:
relaying via the fluid analyzer fluid analysis measurements of fluid in the vent port of the flexible pipe to the recording and processing unit;
monitoring and modeling via the recording and processing unit different types of fluids measured by the fluid analyzer and determining the integrity of the flexible pipe based thereon; and
automating the measuring sequence to recur at predetermined time intervals.

32. The method of claim 23, further comprising controlling a flow of fluid through a pressure and flow measurement system in a regulated or non-regulated manner with a flow controller.

33. The method of claim 32, wherein the flow controller comprises a variable restriction.

34. The method of claim 33, wherein the variable restriction is an electrically controlled valve and further comprising regulating flow through the valve based on a feedback-loop based on a flow meter section reading.

35. The method of claim 32, wherein the flow controller comprises a passive device, including a sonic nozzle having a restriction configured such that fluid flow is accelerated to a critical velocity equal to a local sonic velocity at a throat of the nozzle and leading to a known and constant volumetric flow rate.

36. The method of claim 32, further comprising measuring gas pressure of the annulus with a gas sensor section which is included in the pressure and flow measurement system.

37. The method of claim 36, wherein the gas sensor section is configured to be retrofitted with physical or chemical sensors for detection of fluid components, including H2S and CO2, or to improve flow measurement or interpretation, including sensors for gas density, temperature, and sound velocity.

38. The method of claim 32, further comprising enabling, with the pressure and flow measurement system, collection of vented gas for laboratory analysis with a sample collection port.

39. The method of claim 38, further comprising enabling with the flow controller sample collection at times determined by a user or as automatically programmed into the system either on a periodic basis or triggered by a condition of the annulus changing as detected by a sensor.

40. The method of claim 32, further comprising measuring, with the pressure and flow measurement system, volume/mass flowrate of gas flowing through the system with a volumetric or mass flow meter section.

41. The method of claim 40, wherein the flow meter section comprises volumetric flow meter section, which includes a rotary meter, an ultrasonic meter, or a positive displacement meter comprising a piston meter.

42. The method of claim 40, wherein the flow meter section comprises mass flow metering section which includes a thermal flow meter, or a Coriolis meter.

43. The method of claim 40, wherein the flow meter section comprises a combination of a volumetric meter with a gas pressure or density sensor.

44. The method of claim 32, further comprising detecting water flooding of the annulus of more than one flexible pipe with respective more than one pressure and flow measurement systems coupled to the annulus of the respective more than one flexible pipe.

45. The method of claim 23, further comprising processing a sample of the fluid from the flow line in the inline fluid analyzer.
